(19) United States
(12) Patent Application Publication
(10) Pub. No.: US 2002/0183400 A1
(43) Pub. Date: Dec. 5, 2002

(54) Composition for topical application comprising at least one hydroxystilbene and at least one polyol to solubilize the hydroxystilbene

(75) Inventors: Francine Baldo, Sceaux (FR); Veronique Roger, Bagneux (FR)

Correspondence Address: OBLON SPIVAK MCCLELLAND MAIER & NEUSTADT PC, FOURTH FLOOR, 1755 JEFFERSON DAVIS HIGHWAY, ARLINGTON, VA, 22202, US

(73) Assignee: L'OREAL, Paris (FR)

(21) Appl. No.: 10/078,409

(22) Filed: Feb. 21, 2002

(30) Foreign Application Priority Data
Feb. 21, 2001 (FR) 0102353

(51) Int. Cl. A61K 31/05

(52) U.S. Cl. 514/733

(57) ABSTRACT

The invention concerns a composition suitable for topical application to the skin containing at least one hydroxystilbene and at least one polyol in weight ratio of polyol to hydroxystilbene of at least 150/1.

### FIELD OF THE INVENTION

The present invention relates to a composition suitable for topical application to the skin, comprising, in a physiologically acceptable medium, at least one hydroxystilbene, preferably resveratrol, and at least one polyol.

### BRIEF DISCUSSION OF THE INVENTION

The hydroxystilbenes are compounds corresponding to the general formula (I):

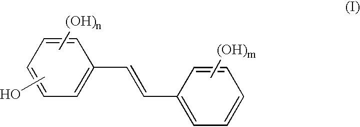

in which n is a whole number between 0 and 4 inclusive and m is a whole number between 0 and 5 inclusive. These compounds may be in a cis or trans form.

According to the invention, the term hydroxystilbene includes the compounds of formula (I) as well as their hydroxyalkyl derivatives.

The hydroxystilbenes are compounds which occur in nature, especially in plants of the spermatophyte class and particularly in vines, grapes and wine.

### DISCUSSION OF THE BACKGROUND

Resveratrol, or 3,4′,5-trihydroxystilbene, is one of the stilbenes which occur in plants, essentially in the spermatophytes, and belong to the class of antibiotic molecules known under the name of phytoalexins.

Resveratrol exists naturally in several plants and fruits in its simple or glucosylated form. The two forms, simple and glucosylated, are in particular found in grape skin (Vrhovsek et al., Am. J. Enol. Vitic., vol. 48, n° 2, 1997) or also in the supernatant of in vitro cultures of Vitis vinifera (Teguo et al., J. Nat. Prod., 61, 655-657,1998).

The resveratrol is liberated in the presence of glucosidases. This reaction occurs naturally in plants, for example in grape skins. During the fermentation of red wines (alcoholic fermentation), this reaction is performed by the glycosidases of the yeasts, but the reaction is not complete and a significant proportion of glucosyl derivatives remains. The glucosylated form is present in varying quantities according to the wine, some varieties of Pinot Noir containing exclusively glucosylated hydroxystilbenes (Soleas et al., Clinical Biochemistry, vol. 30, March 1997).

Various in vitro and in vivo studies have demonstrated the useful biological properties of the hydroxystilbenes, in particular their anti-inflammatory, anti-oxidant and anti-mutagenic properties, and their influence on lipid metabolism and platelet aggregation (Soleas et al., 1997 ; Jang et al., Science, vol. 275, 10 January 1997).

These properties have been exploited in the production of cosmetic compositions containing these compounds.

For example, the international patent application WO 99/04747 discloses cosmetic compositions containing resveratrol, as well as their use for countering skin ageing signs, smoothing the skin or treating wrinkles and fine lines.

### SUMMARY OF THE INVENTION

Despite these useful properties, the hydroxystilbenes, and more particularly resveratrol, have some disadvantages, because of their low solubility in cosmetic solvents. The hydroxystilbenes in fact tend to crystallize. This causes a more or less significant loss of effectiveness of these compounds in the compositions containing them, depending on the degree of crystallisation. In addition, this crystallization can change the overall stability of these compositions and their appearance, which could detract from their attractiveness to users.

The inventors have now discovered that the use of a substantial quantity of polyols, optionally combined with ethanol, avoids the crystallization of the hydroxystilbenes, in particular resveratrol, in all the conventionally used cosmetic media, especially the oil-in-water (O/W) or water-in-oil (W/O) emulsions, the nanoemulsions, the microemulsions, the aqueous gels, the anhydrous gels, the solutions, and the oleosome bases.

### DETAILED DESCRIPTION OF THE INVENTION

By oleosome bases should be understood, within the scope of the present application, emulsions of the oil-in-water type formed from oily globules provided with a lamellar liquid crystal coating, and dispersed in an aqueous phase. These bases are disclosed and claimed in the European patent EP-0 641 557.

A person skilled in the art knows that hydroxystilbenes may be used in cosmetic compositions or for the preparation of cosmetic compositions and/or are suitable for topical application to the skin.

The European patent application EP-0 953 344 discloses the use of an effective quantity of at least one hydroxystilbene as an active component in a composition, or for the preparation of a composition, to encourage the desquamation of the skin, and/or to stimulate the regrowth of the epidermis and/or to counter skin ageing. However, this document does not mention the solubilization of the hydroxystilbene.

Similarly, the international application WO 99/04747 discloses a skin-care composition comprising resveratrol and a cosmetically acceptable vehicle. However, this application does not concern the solubilization of the resveratrol.

The object of the present invention is thus a composition suitable for topical application to the skin comprising, in a physiologically acceptable medium, at least one hydroxystilbene and at least one polyol, in a mass ratio of polyol to hydroxystilbene of at least 150/1.

According to the invention, the hydroxystilbenes may be used alone or in mixtures of any type and may be of natural or synthetic origin.

The hydroxystilbenes which may be used according to the invention include:

4′-hydroxystilbene,

2′,4′-dihydroxystilbene,

3′,4′-dihydroxystilbene,

4,4′-dihydroxystilbene,

2′,4′,4-trihydroxystilbene,

3′,4′,4-trihydroxystilbene,

2,4,4′-trihydroxystilbene,

3,4,4′-trihydroxystilbene,

3,4′,5-trihydroxystilbene,

2′,3,4-trihydroxystilbene,

2,3′,4-trihydroxystilbene,

2′,2,4′-trihydroxystilbene,

2,4,4′,5-tetrahydroxystilbene,

2′,3,4′,5-tetrahydroxystilbene,

2,2′,4,4′-tetrahydroxystilbene,

3,3′,4′,5-tetrahydroxystilbene,

2,3′,4,4′-tetrahydroxystilbene,

3,3′,4,4′-tetrahydroxystilbene,

3,3′,4′,5,5′-pentahydroxystilbene,

2,2′,4,4′,6-pentahydroxystilbene,

2,3′,4,4′,6-pentahydroxystilbene, and

2,2′,4,4′,6,6′-hexahydroxystilbene.

3,4′,5-Trihydroxystilbene, also called resveratrol, is preferably used according to the invention.

The quantity of hydroxystilbene usable according to the invention obviously depends on the effect desired. As an example, the quantity of hydroxystilbene usable according to the invention may vary for example from 0.001% to 10%, and preferably from 0.005% to 0.5% of the total weight of the composition.

The polyols may particularly be selected from glycerine, the glycols, such as mono- or di-propylene glycol, butylene glycol, pentylene glycol, and the polyethylene glycols, in particular containing from 4 to 8 ethylene oxide units, and their mixtures.

The polyols particularly preferred are the polyethylene glycols, in particular polyethylene glycol 8 EO, butylene-1,3-glycol, 5-[2-(4-hydroxyphenyl)vinyl]benzene-1,3-diol and 2-octyidodecanol.

The compositions according to the invention preferably additionally contain an alkanol with from 1 to 6 carbon atoms, in particular ethanol.

The quantity of alkanol present in the composition may reach 10% by weight, preferably 5% by weight with respect to the total weight of the composition.

The composition according to the invention may consist of an emulsion, especially water-in-oil (E/H) or oil-in-water (H/E) or in the form of a multiple emulsion.

The composition according to the invention may also consist of an oil-in-water emulsion formed of oily globules provided with a lamellar liquid crystal coating, and dispersed in an aqueous phase.

Each oily globule, of size less than 500 nanometres and preferably less than 300 nanometres, is coated with a monolamellar or oligolamellar layer obtained from at least one lipophilic surface-active agent, at least one hydrophilic active agent and at least one fatty acid.

By oligolamellar layer should be understood, in the sense of this application, a layer comprising from 2 to 5 lipid lamellas.

The aqueous phase contains the hydroxystilbene in the dissolved state and the solubilizing polyol.

This type of emulsion, also called oleosome base, is disclosed in the European patent EP-0 641557.

The composition according to the invention may contain an oily phase composed of an animal, plant, mineral, silicone, fluorinated and/or synthetic oil.

The oily phase may also contain at least one fatty alcohol or at least one fatty acid, as well as at least one surface-active agent.

Particularly worth mentioning are the hydrocarbon oils such as paraffin oil or vaseline ; perhydrosqualene; shea butter ; arara oil almond, calophyllum, palm, ricin, avocado, jojoba, olive or cereal germ oils; alcohols such as oleic, linoleic or linolenic alcohol, isostearic alcohol or octyl dodecanol.

Also worth mentioning are the silicone oils such as PDMS, optionally phenylated such as the phenyltrimethicones.

Such an ester may in particular be selected from the group consisting of dioctyl adipate, 2-ethylhexyl palmitate, diisopropyl adipate, 2-ethylhexyl hexanoate, ethyl laurate, methyl myristate, octyldodecyl octanoate, isodecyl neopentanoate, ethyl myristate, myristyl propionate, 2-ethylhexyl 2-ethylhexanoate, 2-ethylhexyl octanoate, 2-ethylhexyl caprate/caprylate, methyl palmitate, butyl myristate, isobutyl myristate, ethyl palmitate, isohexyl laurate, hexyl laurate, isopropyl isostearate.

When the composition is an emulsion, the oily phase may be present at a concentration of 5 to 95% of the total weight of the composition.

The composition according to the invention may, in addition, contain:

an agent facilitating the suspension of the fatty phase, for example a copolymer of a C₁₀-C₃₀ alkyl acrylate and acrylic or methacrylic acid or their ester (Pemulen™ TR1, Pemulen™ TR2, Carbopol™ 1342 from GOODRICH) ; or an acrylamidelmethylpropanesulfonic acid copolymer (Sepigel™ from SEPPIC), and/or

an agent facilitating the dispersion of the fatty phase, such as an emulsion or vesicular system based on vesicles, optionally of nanometre size, composed of ionic lipids (liposomes) or non-ionic lipids, and in particular the emulsion systems well known to a skilled person composed of glyceryl stearate/PEG 100 stearate (CTFA), cetyl alcohol and stearyl alcohol, PEG-50 stearate, PEG-40 stearate, sorbitan tristearate, and the stearates of oxyethylenated sorbitan.

The composition of the invention may also contain an agent to modify its viscosity and obtain more or less gelified textures, such as:

the cellulose derivatives (carboxymethylcellulose, hydroxyethylcellulose, hydroxypropylmethylcellulose),

the natural gums such as xanthan, guar, or carob gum, the scleroglucans, derivatives of chitin or chitosan, the carrageenans,

the waxes or gums having for example softening or lubricant properties,

the polycarboxyvinyl derivatives of the Carbomer type (marketed by the GOODRICH company under the trade names CarbopoI™, 940, 951, or by the 3V-SIGMA company under the trade names Synthalen™ K or Synthalen™ L).

The compositions according to the invention may also contain adjuvants currently used in this field, such as preservatives, antioxidants, sequestrants, or gelling agents (especially hydrophilic), perfumes, fillers such as kaolin and starch, or even hollow microspheres, UV filters, skin-care agents, in particular anti-irritant compounds and/or the retinoids and/or the (alpha) hydroxy-acids, and/or vitamins, and/or DHEA derivatives.

Preservatives according to the invention include for example alkylparaben, arylparaben, chlorhexidine derivatives, the alkylbenzoates, salicylic, sorbic and propionic acids, phenoxy ethanol, the alkyl esters and alkali and alkaline earth salts of these acids.

Hydrophilic gelling agents according to the invention include in particular the carboxyvinyl polymers (carbomer), the acrylic copolymers such as the acrylate/alkyl acrylate copolymers, the polyacrylamides, the polysaccharides, the natural gums and clays, and, as lipophilic gelling agents, the modified clays such as the bentonites, the metal salts of fatty acids and hydrophobic silica.

The compositions are most often in the form of a milk, cream, gel or microemulsions, but other methods of presentation are not excluded.

A skilled person will obviously take care that these additional compounds and/or their quantity are selected so that the advantageous properties of the composition according to the invention are not, or not significantly changed by their addition. In particular, these compounds must not impair the advantageous properties of the hydroxystilbene, nor encourage its crystallization.

The composition according to the invention may be used as a skin-care product, or in a make-up product, or as a hair-care product such as a shampoo or conditioner.

The present invention also relates to the cosmetic use of the composition according to the invention for preventing or treating skin-ageing signs.

The present invention also relates to a method for preparing a composition according to the invention, characterized in that it comprises a step consisting of mixing at least one hydroxystilbene with at least one polyol, in a mass ratio of polyol to hydroxystilbene of at least 150/1.

The compositions according to the invention in the form of water-in-oil (W/O), or oil-in-water (O/W), or multiple emulsions, are conventionally prepared by preparation of the aqueous and oily phases and incorporation of one into the other by agitation.

The compositions according to the invention in the form of an oleosome base are prepared as follows:

in a first step, the fatty phase containing the lipophilic surface-active agent, the hydrophilic surface-active agent and the fatty acid, and the aqueous phase containing the basic agent, the hydroxystilbene and the polyol(s) are mixed with agitation, and

in a second step, the mixture obtained is homogenized using the cavitation principle.

In the first step, the mixture is subjected to conventional agitation, for example in a homogenizer rotating at a speed of between about 500 and 5000 r.p.m., for a time of about 10 to 60 min, and at a temperature of between about 20 and 95° C.

In the second step, the homogenization results from the cavitation phenomenon created and maintained within the mixture, then in liquid form, by a linear movement at a speed of at least 100 m/s. It may be performed by use of a high-pressure homogenizer operating at pressures of between about 200 and 1000 bars.

The principle of use of this type of homogenizer is well known to a person skilled in the art. The operation uses successive passages, generally from 2 to 10 passages, under pressure, with the pressure being returned to normal between each passage.

The homogenization of the second step may also be obtained by ultrasound or by use of homogenizers fitted with a head of the rotor-stator type.

If the hydroxystilbene and the polyol(s) are introduced in the free state in the aqueous phase, they are introduced during the first step.

If, however, they are introduced in the encapsulated state in the aqueous phase, they are introduced in a subsequent third step, by simple mixture.

The hydroxystilbene and the polyol(s) are preferably introduced in the free state in the aqueous phase.

The invention will be better illustrated by the following non-limiting examples.

In the examples, except where otherwise stated, all percentages and parts are by weight.

### EXAMPLES

### Products

hydroxystilbenes:

3,4′,5-trihydroxystilbene marketed by the company SIGMA under the name resveratrol™

polyols:

polyethylene glycol (8 EO)

butylene-1,3-glycol

5-[2-(4-hydroxyphenyl)vinyl]benzene-1,3-diol

ethanol

### Emulsions

The solutions of the polyols and the resveratrol were oil-in-water (H/E) emulsions, with and without ethanol, water-in-oil (E/H) emulsions, and oleosome bases.

oil-in-water emulsions (H/E)

5 emulsions E₁, E₂, E₃, E₄ and E₅ whose compositions are given in tables 1 to 5.

water-in-oil emulsions (E/H)

2 emulsions E₆ and E₇ whose compositions are given in tables 6 and 7

oleosome bases

2 emulsions E₈ and E₉ whose compositions are given in tables 8 and 9.

TABLE 1
Emulsion E₁ (O/W)

| Phase | Chemical name | Quantity (%) |
|---|---|---|
| a₁ | Sterilized deionized water | 71.8 |
| | Acrylic acid/stearyl methacrylate copolymer | 0.5 |
| | polymerized in an ethyl acetate/cyclohexane | |
| | mixture | |
| a₂ | Butylene-1,3-glycol | 1 |
| | Methyl p-hydroxybenzoate | 0.2 |
| b | Sterilized deionized water | 2 |
| | Triethanolamine 99% | 0.3 |
| c | Isodecyl neopentanoate | 10 |
| | Propyl p-hydroxybenzoate | 0.1 |
| d | Polyethylene glycol (8 EO) | 5 |
| | Butylene-1,3-glycol | 4 |
| | 5-[2-(4-hydroxyphenyl)vinyl]benzene-1,3-diol | 0.1 |
| | Non-denatured ethyl alcohol 96 degrees | 5 |
| | Non-denatured ethyl alcohol 96 degrees | 5 |

TABLE 2
Emulsion E₂ (O/W)

| Phase | Chemical name | Quantity (%) |
|---|---|---|
| a₁ | Sterilized deionized water | 66.6 |
| | Pentasodium salt of ethylenediamine | 0.1 |
| | tetramethylenephosphonic acid 33% in water, | |
| | unstabilized | |
| | Acrylic acid/stearyl methacrylate copolymer | 0.5 |
| | polymerized in an ethyl acetate/cyclohexane | |
| | mixture | |
| a₂ | Butylene-1,3-glycol | 1 |
| | Methyl p-hydroxybenzoate | 0.2 |
| b | Sterilized deionized water | 2 |
| | Triethanolamine 99% | 0.3 |
| c | Isodecyl neopentanoate | 10 |
| | Propyl p-hydroxybenzoate | 0.1 |
| d | Polyethylene glycol (8 EO) | 7 |
| | Butylene-1,3-glycol | 7 |
| | Butylene-1,3-glycol | 7 |
| | 5-[2-(4-hydroxyphenyl)vinyl]benzene-1,3-diol | 0.2 |
| | 5-[2-(4-hydroxyphenyl)vinyl]benzene-1,3-diol | 0.2 |
| | Non-denatured ethyl alcohol 96 degrees | 5 |
| | Non-denatured ethyl alcohol 96 degrees | 5 |

TABLE 3
Emulsion E₃ (O/W)

| Phase | Chemical name | Quantity (%) |
|---|---|---|
| a₁ | Sterilized deionized water | 71.6 |
| | Pentasodium salt of ethylenediamine | 0.1 |
| | tetramethylenephosphonic acid 33% in water, | |
| | unstabilized | |
| | Acrylic acid/stearyl methacrylate copolymer | 0.5 |
| | polymerized in an ethyl acetate/cyclohexane | |
| | mixture | |
| a₂ | Butylene-1,3-glycol | 1 |
| | Methyl p-hydroxybenzoate | 0.2 |
| b | Sterilized deionized water | 2 |
| | Triethanolamine 99% | 0.3 |
| c | Isodecyl neopentanoate | 10 |
| | Propyl p-hydroxybenzoate | 0.1 |
| d | Polyethylene glycol (8 EO) | 7 |
| | Butylene-1,3-glycol | 7 |
| | 5-[2-(4-hydroxyphenyl)vinyl]benzene-1,3-diol | 0.2 |
| | 5-[2-(4-hydroxyphenyl)vinyl]benzene-1,3-diol | 0.2 |

TABLE 4
Emulsion E₄ (O/W)

| Phase | Chemical name | Quantity (%) |
|---|---|---|
| a | Sorbitan tristearate | 0.9 |
| | Polyethylene glycol (40 EO) stearate | 2 |
| | Pure cetyl alcohol, of natural origin | 4 |
| | Glyceryl mono, di, tri-palmito-stearate | 3 |
| | Myristyl myristate | 2 |
| | 2-Ethylhexyl palmitate | 2 |
| | Hydrogenated isoparaffin (6-8 moles of | 3 |
| | isobutylene) (viscosity: 34 cst at 25° C.) | |
| | 2-Hexyl-1-decyl alcohol | 5 |
| | Propyl p-hydroxybenzoate | 0.15 |
| b | Sterilized deionized water | 43.7 |
| | Methyl p-hydroxybenzoate | 0.25 |
| c | Cyclopentadimethylsiloxane (viscosity: 4 cst) | 10 |
| d | Polyethylene glycol (8 EO) | 9 |
| | Butylene-1,3-glycol | 9 |
| | Butylene-1,3-glycol | 9 |
| | 5-[2-(4-hydroxyphenyl)vinyl]benzene-1,3-diol | 0.2 |
| | 5-[2-(4-hydroxyphenyl)vinyl]benzene-1,3-diol | 0.2 |
| e | Sterilized deionized water | 5 |
| e | Sterilized deionized water | 5 |
| | Imidazolidinyl urea | 0.3 |
| | Imidazolidinyl urea | 0.3 |
| | Polyacrylamidomethylpropanesulfonic acid, | 0.5 |
| | partially neutralized with ammonia and highly | |
| | crosslinked | |
| | Polyacrylamidomethylpropanesulfonic acid, | 0.5 |
| | partially neutralized with ammonia and highly | |
| | crosslinked | |

TABLE 5
Emulsion E₅ (O/W)

| Phase | Chemical name | Quantity (%) |
|---|---|---|
| a | Sorbitan tristearate | 0.9 |
| | Polyethylene glycol (40 EO) stearate | 2 |
| | Pure cetyl alcohol, of natural origin | 4 |
| | Glyceryl mono, di, tri-palmito-stearate | 3 |
| | Myristyl myristate | 2 |
| | 2-Ethylhexyl palmitate | 2 |
| | Hydrogenated isoparaffin (6-8 moles of | 3 |
| | isobutylene) (viscosity: 34 cst at 25° C.) | |
| | 2-Hexyl-1-decyl alcohol | 5 |
| | Propyl p-hydroxybenzoate | 0.15 |
| b | Sterilized deionized water | 45.7 |
| | Methyl p-hydroxybenzoate | 0.25 |
| c | Cyclopentadimethylsiloxane (viscosity: 4 cst) | 10 |
| d | Polyethylene glycol (8 EO) | 8 |
| | Butylene-1,3-glycol | 8 |
| | Butylene-1,3-glycol | 8 |
| | 5-[2-(4-hydroxyphenyl)vinyl]benzene-1,3-diol | 0.2 |
| | 5-[2-(4-hydroxyphenyl)vinyl]benzene-1,3-diol | 0.2 |
| e | Sterilized deionized water | 5 |
| e | Sterilized deionized water | 5 |
| | Imidazolidinyl urea | 0.3 |
| | Imidazolidinyl urea | 0.3 |
| | Polyacrylamidomethylpropanesulfonic acid, | 0.5 |
| | partially neutralized with ammonia and highly | |
| | crosslinked | |
| | linked | |

TABLE 6
Emulsion E₆ (O/W)

| Phase | Chemical name | Quantity (%) |
|---|---|---|
| a | Oxyethylenated | 1.5 |
| | polymethylcetyldimethylmethylsiloxane (20/75- | |
| | 5-viscosity 3000 cst) | |
| | Polyglyceryl isostearate (4 moles) | 0.5 |
| | Isohexadecane | 5 |
| | Octyl-2-dodecanol | 8 |
| | Mixture of acetyl ethylene glycol stearate, | 1 |
| | glyceryl tristearate | |
| | Propyl p-hydroxybenzoate | 0.15 |
| | Deodorized apricot kernel oil (oleic-linoleic | 5 |
| | (66/28) acids triglycerides), refined, | |
| | unstabilized | |
| b | Sterilized deionized water | 67.4 |
| | Methyl p-hydroxybenzoate | 0.25 |
| | Methyl p-hydroxybenzoate | 0.25 |
| | Magnesium sulfate, 7 H₂O | 0.7 |
| | Magnesium sulfate, 7 H₂O | 0.7 |
| | Disodium salt of ethylenediaminetetraacetic | 0.1 |
| | acid, 2 H₂O | |
| | Disodium salt of ethylenediaminetetraacetic | 0.1 |
| | acid,2 H₂O | |
| c | Sterilized deionized water | 5 |
| c | Sterilized deionized water | 5 |
| | Imidazolidinyl urea | 0.3 |
| d | Polyethylene glycol (8 EO) | 5 |
| | 5-[2-(4-hydroxyphenyl)vinyl]benzene-1,3-diol | 0.1 |

TABLE 7
Emulsion E₇ (O/W)

| Phase | Chemical name | Quantity (%) |
|---|---|---|
| a | Oxyethylenated | 1.5 |
| | polymethylcetyldimethylmethylsiloxane (20/75- | |
| | 5-viscosity 3000 cst) | |
| | Polyglyceryl isostearate (4 moles) | 0.5 |
| | Isohexadecane | 5 |
| | Octyl-2-dodecanol | 8 |
| | Mixture of acetyl ethylene glycol stearate, | 1 |
| | glyceryl tristearate | |
| | Propyl p-hydroxybenzoate | 0.15 |
| | Deodorized apricot kernel oil (oleic-linoleic | 5 |
| | (66/28) acids triglycerides), refined, | |
| | unstabilized | |
| b | Sterilized deionized water | 56.3 |
| | Methyl p-hydroxybenzoate | 0.25 |
| | Magnesium sulfate, 7 H₂O | 0.7 |
| | Magnesium sulfate, 7 H₂O | 0.7 |
| | Disodium salt of ethylenediaminetetraacetic | 0.1 |
| | acid, 2 H₂O | |
| | Disodium salt of ethylenediaminetetraacetic | 0.1 |
| | acid, 2 H₂O | |
| c | Sterilized deionized water | 5 |
| c | Sterilized deionized water | 5 |
| | Imidazolidinyl urea | 0.3 |
| | Imidazolidinyl urea | 0.3 |
| d | Polyethylene glycol (8 EO) | 8 |
| d | Polyethylene glycol (8 EO) | 8 |
| | Butylene-1,3-glycol | 8 |
| | 5-[2-(4-hydroxyphenyl)vinyi]benzene-1,3-diol | 0.2 |

TABLE 8
Emulsion E₈ (oleosome base)

| Phase | Chemical name | Quantity (%) |
|---|---|---|
| a | Polyglycerol distearate (2 moles) | 2 |
| | Polyethylene glycol (8 EO) monostearate | 1.35 |
| | Stearic acid (triple pressure) (C₁₆-C₁₈: 50/50) | 1 |
| | Isocetyl stearate | 7 |
| | Refined plant perhydrosqualene | 13 |
| | Di-tert-butyl 4-hydroxytoluene | 0.07 |
| b₁ | Polyethylene glycol (8 EO) | 5 |
| | 5-[2-(4-hydroxyphenyl)vinyl]benzene-1,3-diol | 0.1 |
| b₂ | Sterilized deionized water | 48.68 |
| | Tri-ethanolamine 99% | 0.25 |
| | 2-Phenoxyethanol | 1 |
| | Chlorphenesine | 0.25 |
| | Chlorphenesine | 0.25 |
| | Phenylethyl alcohol | 0.25 |
| | Phenylethyl alcohol | 0.25 |
| | Pentasodium salt of ethylenediamine | 0.05 |
| | tetramethylenephosphonic acid 33% in water, | |
| | unstabilized | |
| | Pentasodium salt of ethylenediamine | 0.05 |
| | tetramethylenephosphonic acid 33% in water, | |
| | unstabilized | |
| c | Sterilized deionized water | 19 |
| | Polyacrylamidomethylpropanesulfonic acid, | 1 |
| | partially neutralized with ammonia and highly | |
| | crosslinked | |

TABLE 9
Emulsion E₉ (oleosome base)

| Phase | Chemical name | Quantity (%) |
|---|---|---|
| a | Polyglycerol distearate (2 moles) | 2 |
| | Polyethylene glycol (8 EO) monostearate | 1.35 |
| | Stearic acid (triple pressure) (C₁₆-C₁₈: 50/50) | 1 |
| | Isocetyl stearate | 7 |
| | Refined plant perhydrosqualene. | 13 |
| | Di-tert-butyl 4-hydroxytoluene | 0.07 |
| b | Sterilized deionized water | 37.58 |
| | Triethanolamine 99% | 0.25 |
| | 2-Phenoxyethanol | 1 |
| | Chlorphenesine | 0.25 |
| | Phenylethyl alcohol | 0.25 |
| | Pentasodium salt of ethylenediamine | 0.05 |
| | tetramethylenephosphonic acid 33% in water, | |
| | unstabilized | |
| c | Sterilized deionized water | 19 |
| | Polyacrylamidomethylpropanesulfonic acid, | 1 |
| | partially neutralized with ammonia and highly | |
| | crosslinked | |
| | Polyacrylamidomethyipropanesulfonic acid, | 1 |
| | partially neutralized with ammonia and highly | |
| | crosslinked | |
| d | Polyethylene glycol (8 EO) | 8 |
| | Polyethylene glycol (8 EO) | 8 |
| | Butylene-1,3-glycol | 8 |
| | Butylene-1,3-glycol | 8 |
| | 5-[2-(4-hydroxyphenyl)vinyl]benzene-1,3-diol | 0.2 |
| | 5-[2-(4-hydroxyphenyl)vinyl]benzene-1,3-diol | 0.2 |

### Example 1

### Solubilization in O/W Emulsion

Resveratrol, in the form and the quantities stated in table 10, was added to emulsions E₁ to E₅.

The physico-chemical stability of the emulsions obtained was verified by macroscopic and microscopic means, after 24 hours and later.

The behaviour of the resveratrol during the solubilization in emulsions E₁ to E₅, and the change overtime, are given in table 10 below.

TABLE 10

| | Physico-chemical stability | | |
|---|---|---|---|
| Emulsions O/W | 24 hours | 1 month | 2 months |
| E₁ + 0.1% pure resveratrol | crystals | — | — |
| [polyols]/[resveratrol] = 100/1 | visible under | | |
| | microscope | | |
| E₂ + 0.2% resveratrol with | — | — | no crystals |
| 52.5% active matter | | | at 4° C. or |
| [polyols]/[resveratrol] = 150/1 | | | 25° C. |
| E₃ + 0.2% | — | crystals at | — |
| [polyols]/[resveratrol] = 150/1 | | 4° C. | |
| E₃ + 0.2% | — | crystals at | — |
| [polyols]/[resveratrol] = 150/1 | | 4° C. | |
| E₄ + 0.2% | — | — | no crystals at |
| [polyols]/[resveratrol] 180/1 | | | 25° C. |
| E₅ + 0.2% resveratrol with | — | — | no crystals at |
| 52.5% active matter | | | 25° C. |
| [polyols]/[resveratrol] = 160/1 | | | |

Table 10 shows that the polyols gave good solubilization of resveratrol in the O/W emulsions, when the mass ratio of polyols to resveratrol is at least 150/1.

The presence of ethanol, combined with the polyols in the composition, further improves the solubilization.

### Example 2

### Solubilization in W/O Emulsion

Resveratrol, in the form and the quantities stated in table 11 was added to emulsions E₆ and E₇.

The physico-chemical stability of the emulsions obtained was, as in example 1 verified by macroscopic and microscopic means, after 24 hours and later.

The behaviour of the resveratrol during the solubilization in emulsions E₆ and E₇, and the change over time, are given in table 11 below.

TABLE 11

| | Physico-chemical stability | |
|---|---|---|
| Emulsions W/O | 24 hours | 2 months |
| E₆ + 0.1% pure resveratrol | crystals at | — |
| [polyols]/[resveratrol] = 50/1 | ambient | |
| | temperature | |
| E₇ + 0.2% resveratrol with | — | no crystals at |
| 52.5% active matter | | 25° C. |
| [polyols]/[resveratrol] = 160/1 | | |

Table 11 shows that the polyols gave good solubilization of resveratrol in W/O emulsions when the mass ratio of polyol to resveratrol was at least 150/1.

### Example 3

### Solubilization in an Oleosome Base

Resveratrol in the form and the quantities stated in table 12 was added to emulsions E₈ and E₉.

The physico-chemical stability of the emulsions obtained was, as in example 1 and 2, verified by macroscopic and microscopic means, after 24 hours and later.

The behaviour of the resveratrol during the solubilization in emulsions E₈ and E₉, and the change over time, are given in table 12 below.

TABLE 12

| | Physico-chemical stability | |
|---|---|---|
| Oleosome base | 1 month | 2 months |
| E₈ + 0.1% pure | crystals at | — |
| resveratrol | 25° C. | |
| [polyols]/[resveratrol] = | | |
| 50/1 | | |
| E₉ + 0.2% de resveratrol | — | no crystals |
| with 52.5% active matter | | at 25° C. |
| [polyols]/[resveratrol] = | | |
| 160/1 | | |

Table 12 shows that the polyols gave good solubilization of resveratrol in the oleosome bases when the mass ratio of the polyols to resveratrol was at least 150/1.

All documents mentioned above are incorporated herein by reference.

French patent application 0102353 filed Feb. 21, 2001, is incorporated herein by reference.

The amount of invention composition to be used varies, and is easily determinable by one of ordinary skill in the art. For example, 0.2-5 g of composition may be applied to, e.g., the face one or more times daily for one or several days or weeks.

The invention composition may be used to treat and/or prevent the signs of ageing, for example skin ageing, and can be used as a skin care, make-up and/or hair care product.

1. A composition comprising at least one hydroxystilbene and at least one polyol, wherein the weight ratio of the polyol to the hydroxystilbene is at least 150/1.

2. The composition according to claim 1, comprising a hydroxystilbene selected from the group consisting of:
4′-hydroxystilbene
2′,4′-dihydroxystilbene,
3′,4′-dihydroxystilbene,
4,4′-dihydroxystilbene,
2′,4′,4-trihydroxystilbene,
3′,4′,4-trihydroxystilbene,
2,4,4′-trihydroxystilbene,
3,4,4′-trihydroxystilbene,
3,4′,5-trihydroxystilbene,
2′,3,4-trihydroxystilbene,
2,3′,4-trihydroxystilbene,
2′,2,4′-trihydroxystilbene,
2,4,4′-tetrahydroxystilbene,
2′,3,4′,5-tetrahydroxystilbene,
2,2′,4,4′-tetrahydroxystilbene,
3,3′,4′,5-tetrahydroxystilbene,
2,3′,4,4′-tetrahydroxystilbene,
3,3′,4,4′-tetrahydroxystilbene,
3,3′,4′,5,5′-pentahydroxystilbene,
2,2′,4,4′,6-pentahydroxystilbene,
2,3′,4,4′,6-pentahydroxystilbene, and
2,2′,4,4′,6,6′-hexahydroxystilbene.

3. The composition according to claim 1, comprising 3,4′,5-trihydroxystilbene.

4. The composition according to claim 1, wherein the at least one hydroxystilbene is present in a quantity of from 0.001% ro 10% by weight with respect to the total weight of the composition.

5. The composition according to claim 1, comprising a polyol selected from the group consisting of glycerine, a glycol, polyethylene glycol and their mixtures.

6. The composition according to claim 5, comprising a polyol selected from the group consisting of polyethylene glycol, butylene-1,3-glycol and 5-[2-(4-hydroxyphenyl)vinyl]benzene-1,3-diol.

7. The composition according to claim 1, further comprising a C₁-C₆, alkanol.

8. The composition according to claim 7, wherein the alkanol represents up to 10% by weight of the total weight of the composition.

9. The composition according to claim 1, wherein said composition is in the form of an emulsion of the oil-in-water type or the water-in-oil type, a nanoemulsion, a microemulsion, an aqueous gel, an anhydrous gel, a solution, or a multiple emulsion.

10. The composition according to claim 1, wherein said composition is in the form of an oil-in-water emulsion formed of oily globules provided with a lamellar liquid crystal coating dispersed in an aqueous phase.

11. The composition according to claim 10, wherein the oily globules have an average diameter of less than 500 nanometers.

12. The composition according to claim 10, wherein the lamellar liquid crystal coating is a monolamellar or oligolamellar layer obtained from at least one lipophilic surface-active agent, at least one hydrophilic surface-active agent, and at least one fatty acid.

13. The composition according to claim 10, wherein the aqueous phase comprises the hydroxystilbene and the polyol in the dissolved state.

14. The composition according to claim 1, further comprising an oily phase, the oily phase comprising animal, plant, mineral, silicone, fluorinated and/or synthetic oil.

15. The composition according to claim 14, wherein the oily phase further comprises at least one fatty alcohol or at least one fatty acid and at least one surface-active agent.

16. The composition according to claim 1, further comprising at least one adjuvant selected from the group consisting of preservatives, perfumes, fillers, UV filters, and skin-care agents.

17. A method comprising mixing at least one hydroxystilbene with at least one polyol in a weight ratio of polyol to hydroxystilbene of at least 150/1.

18. A method for the preparation of and oil-in-water emulsion comprising:
mixing with agitation an oily phase containing a lipophilic surface-active agent, a hydrophilic surface-active agent and a fatty acid with an aqueous phase comprising a basic agent, a polyol and a hydroxystilbene, and
homogenizing the mixture obtained from the first step, wherein the weight ratio of polyol to hydroxystilbene is at least 150/1.

19. The method according to claim 18, wherein the homogenization is achieved either using pressures of between 200 and 1000 bars, or by ultrasound, or by use of homogenizers fitted with a rotor-stator head.

20. A method, comprising applying the composition of claim 1 to the skin in an amount effective to treat and/or prevent signs of skin aging.
